(19) United States
(12) Patent Application Publication
(10) Pub. No.: US 2026/0283608 A1
(43) Pub. Date: Sep. 24, 2026

(54) Adjustable Traction Device for Endoscopic Medical Procedures

(71) Applicants: Justin R. Chambers, Morgantown, WV (US); William T. McClellan, Morgantown, WV (US); M'hamed Turki, Huntington, WV (US); Wesam Frandah, Hurricane, WV (US)

(72) Inventors: Justin R. Chambers, Morgantown, WV (US); William T. McClellan, Morgantown, WV (US); M'hamed Turki, Huntington, WV (US); Wesam Frandah, Hurricane, WV (US)

(21) Appl. No.: 19/573,132

(22) Filed: Mar. 20, 2026

Related U.S. Application Data
(60) Provisional application No. 63/775,454, filed on Mar. 21, 2025.

(51) Int. Cl. A61B 17/02; A61B 17/00

(52) U.S. Cl. CPC A61B 17/0218; A61B 2017/00269; A61B 2017/00681; A61B 2017/00867; A61B 2017/0225

(57) ABSTRACT

A traction device, includes an adjustable tension element having a first end and a second end opposite the first end, and a spring extending between the first end and the second end, wherein: the first end and the second end of the adjustable tension element are configured to be attached to a first tissue and a second tissue, respectively, while the spring is in an expanded configuration; and the spring is configured to exert a traction force between the first tissue and the second tissue responsive to heating the adjustable tension element.

### PRIORITY

This application claims priority to U.S. provisional patent application No. 63/775,454 filed Mar. 21, 2025, the contents of which are incorporated by reference herein in their entirety.

### FIELD OF THE INVENTION

Aspects of the present invention relate generally to medical devices and, more particularly, to an adjustable traction device for endoscopic medical procedures.

### BACKGROUND

Submucosal dissection refers to a surgical technique primarily used in endoscopic procedures to remove lesions from the gastrointestinal (GI) tract, particularly in the esophagus, stomach, and colon. The most well-known application is Endoscopic Submucosal Dissection (ESD), which was developed as an advanced method to achieve en bloc resection of superficial tumors, avoiding more invasive surgeries.

Submucosal dissection, particularly in endoscopic procedures like ESD, presents several challenges and complications that can impact its success and safety. One of the primary difficulties is the technical complexity of the procedure, requiring extensive training and experience due to the precise manipulation of specialized endoscopic knives in the delicate submucosal layer. The risk of perforation is a significant concern, especially in thinner-walled organs like the esophagus and colon, where even minor miscalculations can lead to full-thickness tears. Bleeding is another frequent complication, as the submucosal layer is rich in blood vessels, necessitating meticulous hemostatic techniques to prevent intraoperative and delayed bleeding. Furthermore, post-procedural fibrosis and stricture formation can occur, particularly in esophageal ESD, leading to dysphagia and requiring additional interventions such as balloon dilation. The lengthy procedure time compared to other endoscopic resections also poses a challenge, increasing patient discomfort and procedural fatigue for endoscopists. One of the most significant challenges in particular is lifting the tumor as it is dissected to allow proper visualization as the resection is being performed.

### SUMMARY

Various implementations of the invention address the above-noted problems of the prior art by providing an inventive device and method to apply adjustable traction/tension to a tumor during an endoscopic resectioning procedure such as ESD. Currently, traction can be applied via rubber bands, springs, sutures, etc., but these technique do not offer the ability to adjust the traction/tension throughout the procedure. The challenges of not having adjustment during the procedure causes most traction techniques to be complicated and time consuming to utilize. The ability to apply the device and adjust traction as needed throughout the procedure allows for significant improvement over current traction techniques. Therefore, a novel device and method to apply adjustable traction to a tissue during resection procedures can reduce procedure time, reduce the risk of perforation, and improve the overall outcomes

In accordance with an aspect of the invention, a traction device includes an adjustable tension element having a first end and a second end opposite the first end, and a spring extending between the first end and the second end, wherein the first end and the second end of the adjustable tension element are configured to be attached to a first tissue and a second tissue, respectively, while the spring is in an expanded configuration, the spring is configured to exert a traction force between the first tissue and the second tissue responsive to heating the adjustable tension element.

In embodiments, the traction device further comprises a first loop at the first end of the adjustable tension element, and a second loop at the second end of the adjustable tension element.

In some embodiments, the spring is composed of a shape changing material. In one example, the shape changing material comprises Nitinol. In embodiments, the spring composed of the shape changing material has a programmed shape that defines a contracted configuration. In one example, the spring has a first length in the contracted configuration, the spring has a second length in the expanded configuration, and the first length is less than the second length. In accordance with aspects of the invention, the heating the adjustable tension element comprises heating the shape changing material of the spring, and the spring is configured to change shape from the expanded configuration to the contracted configuration responsive to heating the shape changing material of the spring beyond a transition temperature of the shape changing material. In some implementations, a transition temperature of the shape changing material is greater than 100° F. In embodiments, the spring comprises a helical spring, wherein an inner diameter of coils of the helical spring corresponds to an outer diameter of a hemostatic clip delivery device such that the hemostatic clip delivery device is capable of being passed through and in contact with the coils of the helical spring.

In accordance with aspects of the invention, the first end of the adjustable tension element is configured to be attached to the first tissue using a first hemostatic clip while the first hemostatic clip engages the first loop at the first end of the adjustable tension element, and the second end of the adjustable tension element is configured to be attached to the second tissue using a second hemostatic clip while the second hemostatic clip engages the second loop at the second end of the adjustable tension element.

In some embodiments, the traction device comprises a first clamp connected to the first loop at the first end of the adjustable tension element, and a second clamp connected to the second loop at the second end of the adjustable tension element.

In some embodiments, the adjustable tension element is moveable between the expanded configuration and a contracted configuration, and further comprising a locking mechanism that resists movement of the adjustable tension element from the contracted configuration to the expanded configuration.

In some embodiments, the adjustable tension element comprises a sacrificial material configured to hold the spring in the expanded configuration, and the sacrificial material is configured to disintegrate and permit the spring to contract responsive to the heating of the adjustable tension element.

In accordance with further aspects of the invention, a method of using the traction device comprises: attaching the first end to the first tissue using a first clamp; attaching the second end to the second tissue using a second clamp; and applying the heating to the adjustable tension element while the first end and the send end are attached to the first tissue and the second tissue, respectively. The method may further comprise: cutting on or around the first tissue or the second tissue after applying the heating and while the first end and the second end are attached to the first tissue and the second tissue, respectively; and applying another heating to the to the adjustable tension element after the cutting and while the first end and the second end are attached to the first tissue and the second tissue, respectively.

In accordance with aspects of the invention, there is a method of applying traction during a procedure, the method comprising: attaching a first end of an adjustable tension element of a traction device to a first tissue using a first clamp, wherein the adjustable tension element comprises a helical spring composed of a shape changing material; attaching a second end of the adjustable tension element to a second tissue using a second clamp while the first end of the adjustable tension element is attached to the first tissue; and causing a traction force to be exerted between the first tissue and the second tissue by heating a first portion of the helical spring to a temperature above a transition temperature of the shape changing material. The method may further comprise: cutting on or around the first tissue or the second tissue after applying the heating and while the first end and the send end are attached to the first tissue and the second tissue, respectively; and adjusting the traction force exerted between the first tissue and the second tissue by heating a second portion of the helical spring to a temperature above the transition temperature of the shape changing material. In embodiments of the method, the attaching the first end to the first tissue using the first clamp comprises using a first hemostatic clip to simultaneously engage the first tissue and a first loop at the first end of the adjustable tension element, and the attaching the second end to the second tissue using the second clamp comprises using a second hemostatic clip to simultaneously engage the second tissue and a second loop at the second end of the adjustable tension element.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE SEVERAL VIEWS OF THE DRAWINGS

Aspects of the present invention are described in the detailed description which follows, in reference to the noted plurality of drawings by way of non-limiting examples of exemplary embodiments of the present invention. In the drawings, the same reference characters may refer to the same or similar parts throughout the different views.

FIG. 1A-1L show an exemplary method of using an adjustable traction device in accordance with aspects of the invention.

FIGS. 2A and 2B shows additional aspects of the adjustable traction device in accordance with aspects of the invention.

FIG. 3 shows an embodiment of the adjustable traction device in accordance with aspects of the invention.

FIGS. 4A and 4B show an embodiment of the adjustable traction device in accordance with aspects of the invention.

FIG. 5 shows another embodiment of an adjustable traction device in accordance with aspects of the invention.

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details in more detail than is necessary for the fundamental understanding of aspects of the present invention, the description taken with the drawings making apparent to those skilled in the art how several forms of the present invention may be embodied in practice.

In accordance with aspects of the invention, an adjustable traction device comprises an adjustable tension element composed of a shape-changing material. The adjustable traction device may be used in medical procedures to remove lesions from the gastrointestinal (GI) tract, such as Endoscopic Submucosal Dissection (ESD) in the esophagus, stomach, or colon. In embodiments, when opposite ends of the adjustable traction device are connected to tissue, an amount of traction force applied to the tissue by the adjustable traction device is selectively adjustable by applying external energy to adjustable tension element, which changes shape in response to the application of the energy. In a preferred embodiment, the adjustable tension element comprises a helical (e.g., coil) spring that is composed of a shape changing material that is austenitic above a transition temperature and martensitic below the transition temperature. In the preferred embodiment, the adjustable tension element has a programmed shape (also called a memory shape) having a first length between ends of the spring, and the adjustable tension element may be stretched (e.g., deformed) to a second shape having a second length that is longer than the first length when the temperature of the adjustable tension element is less than the transition temperature. Due to its shape changing material, the adjustable tension element is configured to return to the first shape responsive to the temperature of the adjustable tension element exceeding the transition temperature. In this manner, the adjustable traction device may be connected between two attachment points of tissue when the adjustable tension element is in the second shape, and the adjustable traction device may then be used to selectively apply tension between the attachment points of the tissue by heating the adjustable tension element above the transition temperature, e.g., because heating the adjustable tension element causes the adjustable tension element to act as a tension spring that shortens (e.g., contracts) between the attachment points of tissue as the adjustable tension element returns to its first shape.

In various embodiments, the adjustable tension element is composed of Nitinol, which is a shape changing alloy composed of nickel and titanium. In a preferred embodiment, the transition temperature of the material of the adjustable tension element is sufficiently high that it will not be reached by heating due to the human body and sufficiently low that it minimizes the risk of burning the tissue. In one example, the transition temperature of the material of the adjustable tension element is in a range of 115° F. to 125° F.

In embodiments, an external energy source is used to selectively apply energy to the adjustable tension element for heating the adjustable tension element above its transition temperature. In one example, energy for heating the adjustable tension element above the transition temperature may be applied to the adjustable tension element using an electrocautery device that is used to dissect tissue during the ESD procedure, e.g., by bringing the electrocautery device into contact (or close proximity) with the adjustable tension element. In this example, the adjustable tension element may be provided with a flux coating that is configured to improve the energy transfer from the electrocautery device to the adjustable tension element (e.g., with the electrocautery device forming an arc or plasma which creates local heating of the material of the adjustable tension element). In another example, energy for heating the adjustable tension element above the transition temperature may be applied to the adjustable tension element using a radiative and/or conductive heater, such as a resistive heating element, in particular a ceramic heating element, for example. In this example, a temperature of the resistive heating element may be controlled (e.g., manually and/or electronically) to cycle between a high temperature (e.g., 140° F.) and a low temperature (e.g., 100° F.) to reduce the risk of the heating element burning the tissue when applying energy to the adjustable tension element. The high temperature of the cycle may be higher than the transition temperature of the of the material of the adjustable tension element and the low temperature of the cycle may be lower than the transition temperature of the material of the adjustable tension element. Other types of heaters may also be used to selectively apply energy to the adjustable tension element.

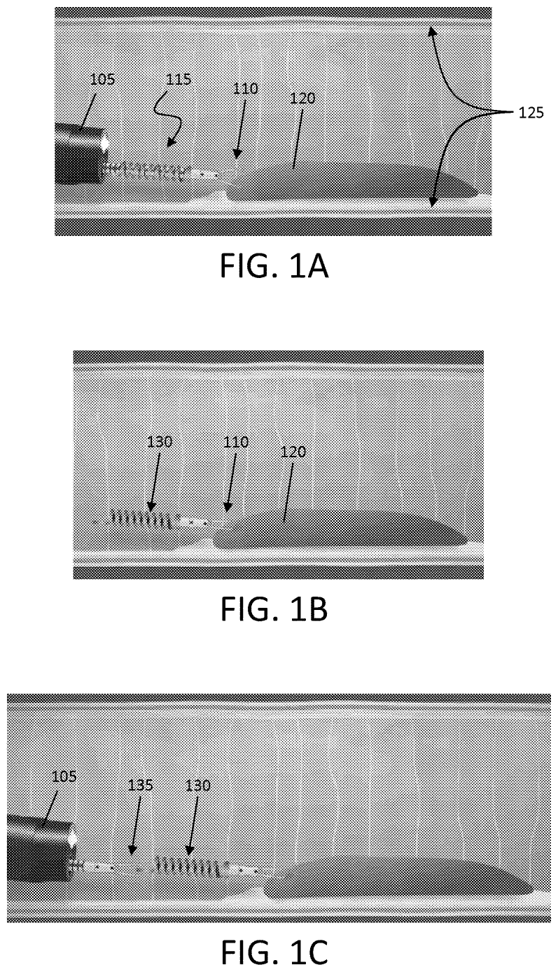

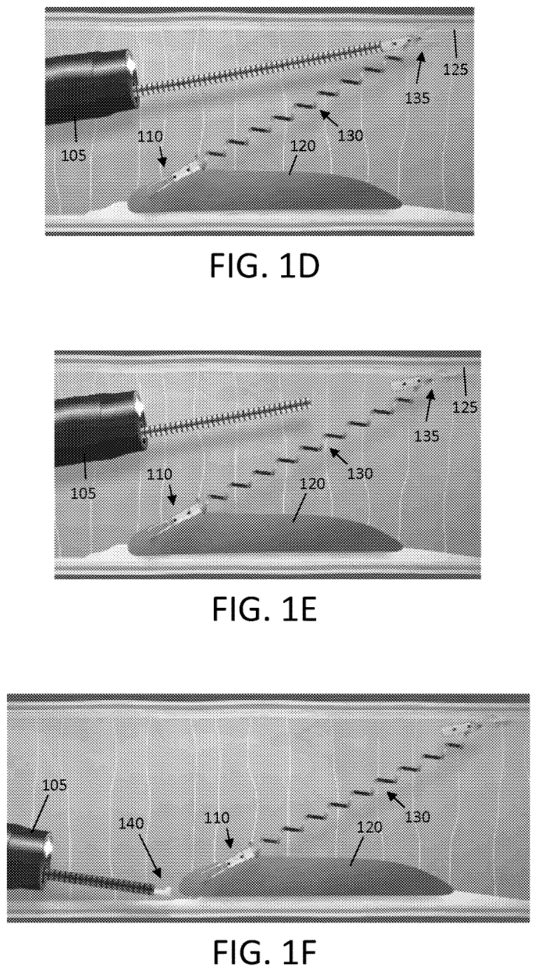

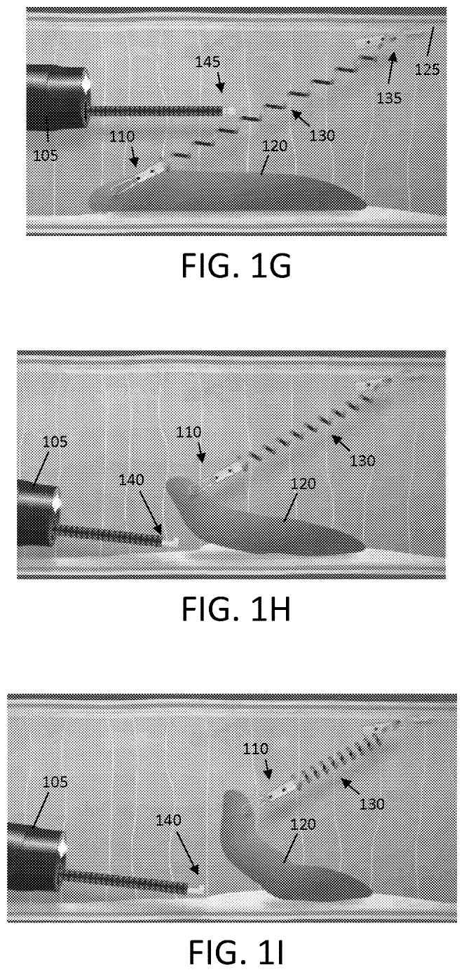

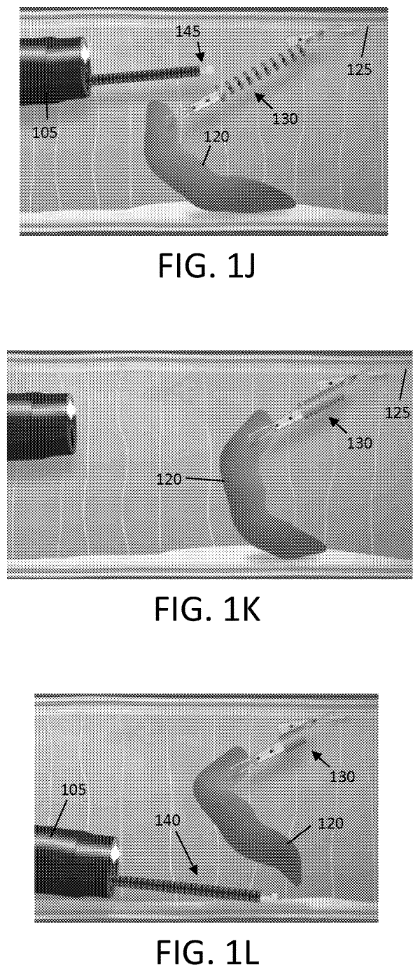

FIG. 1A-1L show an exemplary method of using an adjustable traction device in accordance with aspects of the invention. In FIG. 1A, an endoscope 105 is used to position a first clamp 110 at first end of the adjustable traction device 115 proximate to a first tissue 120. In this example, the first tissue 120 comprises a lesion, such as a tumor, on a second tissue 125 such as a bowel wall.

In FIG. 1B, the endoscope (not shown) attaches the first clamp 110 to the first tissue 120 and detaches the first clamp 110 from the endoscope. In embodiments, the first clamp 110 comprises a hemostatic clip that engages both the first tissue 120 and a first end of the adjustable tension element 130. The hemostatic clip may be broken away from a delivery tool in the endoscope after the hemostatic clip has been attached to the first tissue 120.

In FIG. 1C, the endoscope 105 grasps the second end of the adjustable tension element 130 using a second clamp 135. The second clamp 135 may comprise a hemostatic clip that may be broken away from a delivery tool in the endoscope after the hemostatic clip has been clamped shut.

In FIG. 1D, the endoscope 105 moves the second clamp 135 to the bowel wall 125 at a location spaced apart from the first tissue 120 while the first clamp 110 is clamped to the first tissue 120. This movement of the second clamp 135, which is engaged with the second end of the adjustable tension element 130, causes a stretching of the adjustable tension element 130 to a second length that is longer than the first length of its programmed shape. The adjustable tension element 130 is capable of stretching (e.g., deforming) in this manner, without putting appreciable tension on the first tissue 120, due to the shape changing material of the adjustable tension element 130 being at a temperature below its transition temperature.

In FIG. 1E, the endoscope 105 clamps the second clamp 135 to the second tissue 125 and disconnects the second clamp 135 from the endoscope 105. As shown in FIG. 1E, the adjustable tension element 130 is stretched between the first clamp 110 attached to the first tissue 120 and the second clamp 135 attached to the second tissue 125. In some embodiments, the adjustable tension element 130 comprises respective loops at its opposite ends that are engaged by the respective clamps 110 and 135.

In FIG. 1F, a cutting tool 140 of the endoscope 105 is used to begin cutting the first tissue 120 from the bowel wall. The cutting tool 140 may comprise an electrocautery device, for example. In embodiments, and as shown in FIG. 1F, only a small cut is initially made at this step.

In FIG. 1G, an energy emitting tool 145 of the endoscope 105 is used to selectively heat a portion of the adjustable tension element 130 to a temperature higher than the transition temperature of the shape changing material of the adjustable tension element 130. In this example, the energy emitting tool 145 comprises a ceramic heater that applies energy to the adjustable tension element 130 via radiation and/or conduction. In accordance with aspects of the invention, heating the portion of the adjustable tension element 130 to a temperature higher than the transition temperature causes that portion of the adjustable tension element 130 to contract toward the programmed shape. In this example, because the adjustable tension element 130 is connected as a tension spring between the first tissue 120 and the second tissue 125, this contraction of a portion of the adjustable tension element 130 increases the tension spring force between the first tissue 120 and the second tissue 125, thereby urging the first tissue 120 away from the bowel wall to which it is attached.

In FIG. 1H, the cutting tool 140 of the endoscope 105 is used to continue cutting the first tissue 120 from the bowel wall. In accordance with aspects of the invention, the adjustable tension element 130 contracts and pulls the first tissue 120 away from the bowel wall as this cutting occurs, e.g., due to the shape changing of the portion of the adjustable tension element 130 that occurs as a result of the heating in the previous step.

In FIG. 1I, the cutting with the cutting tool 140 continues and the first tissue 120 is further pulled away from the bowel wall due to the traction force applied by the adjustable tension element 130.

In FIG. 1J, the energy emitting tool 145 of the endoscope 105 is again used to selectively heat another (e.g., second) portion of the adjustable tension element 130 to a temperature higher than the transition temperature of the shape changing material of the adjustable tension element 130. In accordance with aspects of the invention, heating the second portion of the adjustable tension element 130 to a temperature higher than the transition temperature causes that second portion of the adjustable tension element 130 to contract toward the programmed shape. This contraction of the second portion of the adjustable tension element 130, illustrated in FIG. 1K, increases the tension spring force between the first tissue 120 and the second tissue 125, thereby urging the first tissue 120 away from the bowel wall to which it is attached.

In FIG. 1L, the cutting tool 140 of the endoscope 105 is used to continue cutting the first tissue 120 from the bowel wall until the first tissue 120 is completely detached from the bowel wall. In accordance with aspects of the invention, the adjustable tension element 130 contracts and pulls the first tissue 120 away from the bowel wall as this cutting occurs, e.g., due to the shape changing of the portion of the adjustable tension element 130 that occurs as a result of the heating in the previous steps.

The steps illustrated in FIG. 1A-1L thus provide a method for providing adjustable traction, the method comprising: deploying the adjustable traction device 115 inside of the bowel through the normal endoscope used in the procedure the adjustable traction device 115 including the adjustable tension element 130; attaching respective ends of the adjustable tension element 130 to the tumor and bowel wall using clips or other attachment techniques; and adjusting the tension of the adjustable tension element 130 throughout the procedure using an energy source in the form of the energy emitting tool 145. A user such as a surgeon may control the endoscope to perform the steps of the method.

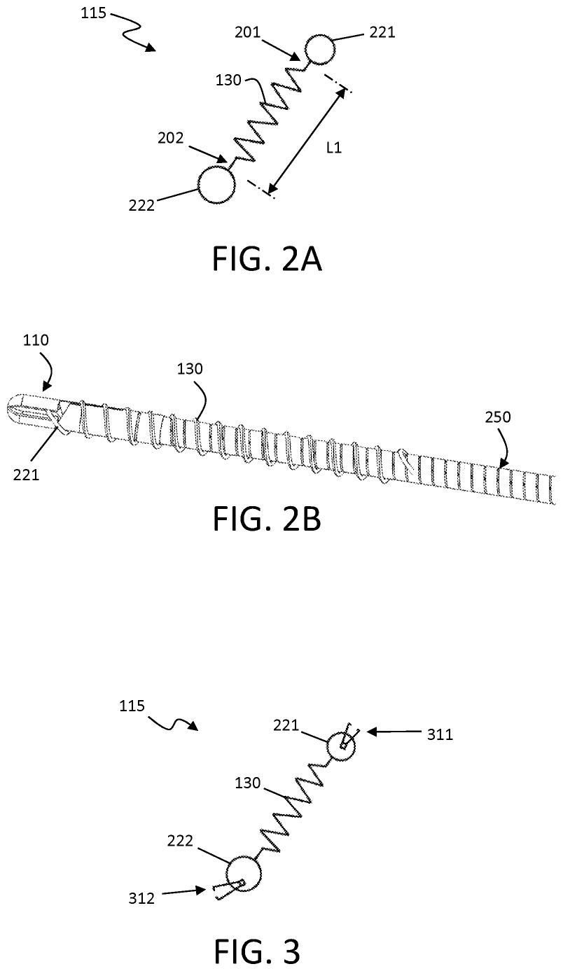

FIGS. 2A and 2B shows additional aspects of the adjustable traction device 115 in accordance with aspects of the invention. As shown in FIG. 2A, the adjustable traction device 115 may include the adjustable tension element 130 in the form of a helical spring composed of a shape changing material (e.g., Nitinol). In various embodiments, the spring is formed of coiled wire that has a rounded shape in cross section (e.g., circular, oval, elliptical, etc.). In various embodiments, the spring has a first shape having a first length L1 between respective ends 201, 202 of the spring. In embodiments, the spring may be deformed from its first shape to a second shape, having a second length longer than the first length L1, when the material of the spring has a temperature that is less than the transition temperature of the material. In embodiments, the spring retains the second shape while the temperature of the material remains below the transition temperature, and the spring will change shape from the second shape back to the first shape responsive to the temperature of the material being raised above the transition temperature. In some embodiments, the spring may include end terminations in the form of loops 221 and 222 at the respective ends 201, 202 of the spring. These loops 221, 222 may be engaged by clamps for attaching the ends of the adjustable traction device 115 to tissue.

FIG. 2B shows an example of a configuration for attaching a first end of the adjustable tension element 130 to a first tissue (e.g., as in FIGS. 1A and 1B) in accordance with aspects of the invention. In this example, the adjustable tension element 130 is coiled around the exterior of a hemostatic clip delivery device 250 that includes a hemostatic clip that forms the first clamp 110. In embodiments, the coil diameter of the spring of the adjustable tension element 130 is such that the spring can fit snuggly around the hemostatic clip delivery device and still allow the hemostatic clip delivery device and the adjustable tension element 130 to pass through a port in the endoscope 105. In one example, the hemostatic clip delivery device 250 comprises a long, flexible catheter-handle assembly that is passed through the working channel of an endoscope and that allows for the positioning, rotation, and deployment of a hemostatic clip under direct endoscopic visualization. In various embodiments, the spring of the adjustable tension element 130 is designed to work universally with many hemostatic clips on the market and uses the loops 221, 222 to be loaded into the jaws of the clip (e.g., without being not directly coupled to the clip). In some embodiments, the spring of the adjustable tension element 130 is pre-stretched to the second length after loading the spring onto the hemostatic clip delivery device and prior to attaching the first clamp to the first tissue.

FIG. 3 shows an embodiment of the adjustable traction device 115 in accordance with aspects of the invention. In this embodiment, the adjustable traction device 115 includes the elements shown in FIG. 2A and additionally includes first and second clamps 311 and 312 connected to the loops 221 and 222, respectively. The clamps 311 and 312 may comprise any conventional or later developed clamp that is capable of being connected to one of the loops and used to clamp onto tissue. In this manner, the adjustable traction device 115 may include the adjustable tension element 130 and the clamps 311 and 312.

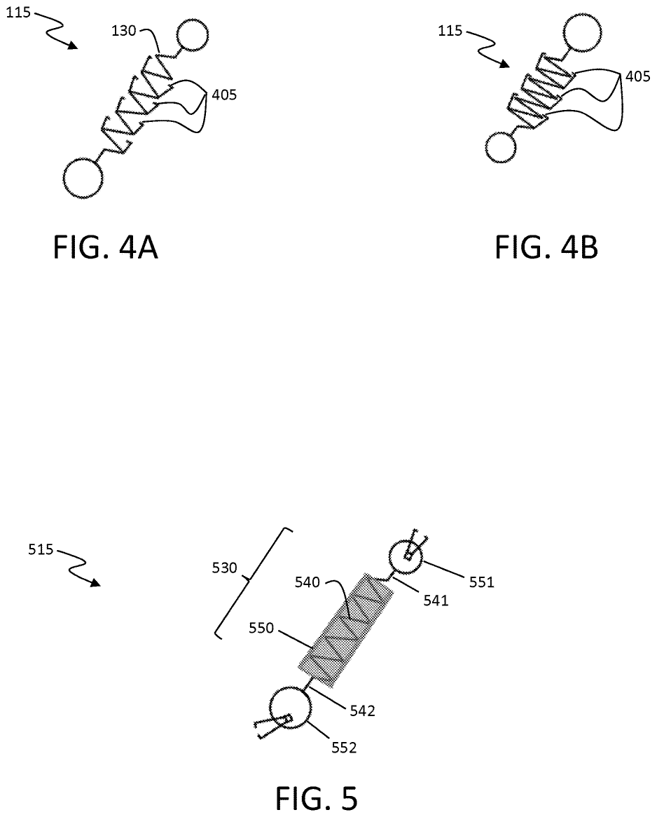

FIGS. 4A and 4B show an embodiment of the adjustable traction device 115 in accordance with aspects of the invention. In this embodiment, the adjustable traction device 115 includes the elements shown in FIG. 2A and additionally includes a locking mechanism on the adjustable tension element 130. In embodiments, the locking mechanism comprises a one-way ratchet mechanism that resists movement of the spring from the first shape to the second shape. In one example, the one-way ratchet mechanism comprises flanges 405 on the spring that engage portions of the spring when the spring is in the first shape. In this example, the flanges 405 resist expansion of the spring from the first shape to a longer second shape. In this example, the locking mechanism is configured such that the resist force provided by the flanges 405 may be overcome by application of a sufficiently large stretching force applied by a user purposefully stretching the spring, but the resist force itself is of a magnitude that it will not be overcome by gravity or by a traction force encountered during an ESD procedure, e.g., as in FIG. 1H-1L. In this manner, a user may apply force to stretch the spring to the first shape (e.g., as shown in FIG. 4A) and then during use as the spring contracts due to the application of energy (e.g., as in FIG. 1H-1L), the locking mechanism retains the spring in the contracted shape (e.g., as shown in FIG. 4B).

FIG. 5 shows another embodiment of an adjustable traction device 515 in accordance with aspects of the invention. In this embodiment, the adjustable traction device 515 comprises an adjustable tension element 530 in the form of a helical spring 540 with end terminations in the form of loops 541 and 542 at the respective ends 551 and 552 of the spring 540. In this embodiment, the spring 540 is a tension spring composed of a non-shape changing material such as stainless steel. In this embodiment, the adjustable tension element 530 comprises a sacrificial material 550 that holds the spring 540 in a second shape that has a second length. In one example, the sacrificial material 550 comprises wax or another substance that is solid at human body temperature and that may be disintegrated (e.g., melted) by applying heat to the material. In embodiments, the adjustable tension element 530 is configured such that the spring 540 is pre-stretched to the second length, the sacrificial material 550 is embedded around the spring 540 to hold the spring 540 in the second length, and the sacrificial material 550 may later be melted to release the spring to contract from the second length to the first length that is shorter than the second length. In this manner, the two ends of the adjustable traction device 515 may be attached to two tissue locations while the sacrificial material 550 is in place around the spring 540, and then the energy source may be used to apply heat to the sacrificial material 550 to melt the sacrificial material 550, thereby permitting the spring to contract to the first length and create a traction force between the two tissue locations. The adjustable traction device 515 may include clamps 561 and 562 connected to the loops 541 and 542, respectively, or may be used with clamps in the form of hemostatic clips as described herein.

Additional aspects of the invention include manufacturing and/or using a adjustable traction device as described herein. Even further aspects of the invention include providing instructions for using a adjustable traction device as described herein. The instructions may be at least one of printed and video.

It is noted that the foregoing examples have been provided merely for the purpose of explanation and are in no way to be construed as limiting of implementations of the present invention. While aspects of the present invention have been described with reference to an exemplary embodiment, it is understood that the words which have been used herein are words of description and illustration, rather than words of limitation. Changes may be made, within the purview of the appended claims, as presently stated and as amended, without departing from the scope and spirit of the present disclosure in its aspects. Although implementations of the present invention have been described herein with reference to particular means, materials and embodiments, implementations of the present invention are not intended to be limited to the particulars disclosed herein; rather, implementations of the present invention extend to all functionally equivalent structures, methods and uses, such as are within the scope of the appended claims.

Features which are described in the context of separate aspects and embodiments of the invention may be used together and/or be interchangeable unless the context clearly dictates otherwise. Similarly, features described in the context of a single embodiment may also be provided separately or in any suitable sub-combination unless the context clearly dictates otherwise.

What is claimed:

1. A traction device, comprising:
an adjustable tension element having a first end and a second end opposite the first end, and a spring extending between the first end and the second end, wherein:
the first end and the second end of the adjustable tension element are configured to be attached to a first tissue and a second tissue, respectively, while the spring is in an expanded configuration; and
the spring is configured to exert a traction force between the first tissue and the second tissue responsive to heating the adjustable tension element.

2. The traction device of claim 1, further comprising:
a first loop at the first end of the adjustable tension element; and
a second loop at the second end of the adjustable tension element.

3. The traction device of claim 1, wherein the spring is composed of a shape changing material.

4. The traction device of claim 3, wherein the shape changing material comprises Nitinol.

5. The traction device of claim 3, wherein the spring composed of the shape changing material has a programmed shape that defines a contracted configuration.

6. The traction device of claim 5, wherein:
the spring has a first length in the contracted configuration;
the spring has a second length in the expanded configuration; and
the first length is less than the second length.

7. The traction device of claim 6, wherein the heating the adjustable tension element comprises heating the shape changing material of the spring.

8. The traction device of claim 6, wherein the spring is configured to change shape from the expanded configuration to the contracted configuration responsive to heating the shape changing material of the spring beyond a transition temperature of the shape changing material.

9. The traction device of claim 3, wherein a transition temperature of the shape changing material is greater than 100° F.

10. The traction device of claim 3, wherein the spring comprises a helical spring.

11. The traction device of claim 10, wherein an inner diameter of coils of the helical spring corresponds to an outer diameter of a hemostatic clip delivery device such that the hemostatic clip delivery device is capable of being passed through and in contact with the coils of the helical spring.

12. The traction device of claim 2, wherein:
the first end of the adjustable tension element is configured to be attached to the first tissue using a first hemostatic clip while the first hemostatic clip engages the first loop at the first end of the adjustable tension element; and
the second end of the adjustable tension element is configured to be attached to the second tissue using a second hemostatic clip while the second hemostatic clip engages the second loop at the second end of the adjustable tension element.

13. The traction device of claim 2, further comprising:
a first clamp connected to the first loop at the first end of the adjustable tension element; and
a second clamp connected to the second loop at the second end of the adjustable tension element.

14. The traction device of claim 2, wherein the adjustable tension element is moveable between the expanded configuration and a contracted configuration, and further comprising a locking mechanism that resists movement of the adjustable tension element from the contracted configuration to the expanded configuration.

15. The traction device of claim 2, wherein:
the adjustable tension element comprises a sacrificial material configured to hold the spring in the expanded configuration; and
the sacrificial material is configured to disintegrate and permit the spring to contract responsive to the heating of the adjustable tension element.

16. A method of using the traction device of claim 1, comprising:
attaching the first end to the first tissue using a first clamp;
attaching the second end to the second tissue using a second clamp;
applying the heating to the adjustable tension element while the first end and the send end are attached to the first tissue and the second tissue, respectively.

17. The method of claim 16, further comprising:
cutting on or around the first tissue or the second tissue after applying the heating and while the first end and the second end are attached to the first tissue and the second tissue, respectively; and
applying another heating to the to the adjustable tension element after the cutting and while the first end and the second end are attached to the first tissue and the second tissue, respectively.

18. A method of applying traction during a procedure, comprising:
attaching a first end of an adjustable tension element of a traction device to a first tissue using a first clamp, wherein the adjustable tension element comprises a helical spring composed of a shape changing material;
attaching a second end of the adjustable tension element to a second tissue using a second clamp while the first end of the adjustable tension element is attached to the first tissue; and
causing a traction force to be exerted between the first tissue and the second tissue by heating a first portion of the helical spring to a temperature above a transition temperature of the shape changing material.

19. The method of claim 18, further comprising:
cutting on or around the first tissue or the second tissue after applying the heating and while the first end and the send end are attached to the first tissue and the second tissue, respectively; and
adjusting the traction force exerted between the first tissue and the second tissue by heating a second portion of the helical spring to a temperature above the transition temperature of the shape changing material.

20. The method of claim 18, wherein:
the attaching the first end to the first tissue using the first clamp comprises using a first hemostatic clip to simultaneously engage the first tissue and a first loop at the first end of the adjustable tension element; and
the attaching the second end to the second tissue using the second clamp comprises using a second hemostatic clip to simultaneously engage the second tissue and a second loop at the second end of the adjustable tension element.
